(19) United States
(12) Patent Application Publication
(10) Pub. No.: US 2026/0263043 A1
(43) Pub. Date: Sep. 10, 2026

(54) INTRACARDIAC ECHOCARDIOGRAPHY (ICE) CATHETER HANDLE WITH 4D ICE CONTROL

(71) Applicant: yoR Labs, Inc., Beaverton, OR (US)

(72) Inventors: Anshumali Roy, Portland, OR (US); Dakota Pellegrino, Hillsboro, OR (US)

(21) Appl. No.: 19/560,762

(22) Filed: Mar. 9, 2026

Related U.S. Application Data
(60) Provisional application No. 63/769,612, filed on Mar. 10, 2025.

(51) Int. Cl. A61B 8/00; A61B 8/08; A61B 8/12

(52) U.S. Cl. CPC A61B 8/467; A61B 8/0883; A61B 8/12; A61B 8/488

(57) ABSTRACT

An intracardiac echocardiography (ICE) catheter handle with integrated controls can be used for 4D ICE, enabling real-time ultrasound image manipulation directly from the handle. Traditional systems require a separate sonographer, causing workflow inefficiencies. This design embeds programmable buttons, allowing cardiac interventionists to adjust imaging planes, rotation, translation, and depth without third-party assistance. It also features dedicated controls for switching imaging modes, including B-mode, color Doppler, and pulse-wave Doppler, ensuring seamless transitions. The system can integrate via wired or wireless configurations to a user-interface screen where the imaging results of the scan can be shown. The device can improve procedural workflow and enhances physician autonomy by eliminating delays associated with verbal communication. Additionally, this innovation streamlines imaging operations, minimizes clutter in the operating room, and facilitates more efficient clinical decision-making in ICE-guided procedures.

### INCORPORATION BY REFERENCE TO ANY PRIORITY APPLICATIONS

Any and all applications for which a foreign or domestic priority claim is identified in the Application Data Sheet as filed with the present application are hereby incorporated in their entireties by reference under 37 CFR 1.57. In particular, this application claims priority to the U.S. Provisional Application 63/769612, filed Mar. 10, 2025, which is incorporated by reference herein in its entirety as if fully set forth herein.

### TECHNICAL FIELD

The present disclosure relates to the field of intracardiac echocardiography (ICE), and more specifically to a catheter handle with integrated controls for 4D ICE imaging, enabling real-time volumetric ultrasound image manipulation directly from the ICE catheter handle during a procedure.

### SUMMARY

For purposes of summarizing, certain aspects, advantages, and novel features have been described herein. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested herein.

With the continuous expansion of the medical device industry, the demand for innovative devices capable of processing and providing real-time diagnostic insights has increased. The IntraCardiac Echocardiography (ICE) systems and methods described herein enable more efficient real-time volumetric imaging of cardiac structures, eliminating the need for a third-party operator to adjust imaging parameters. Such improvements could streamline workflows and enhance procedural control for cardiac interventionists.

The disclosure herein includes a catheter handle with integrated controls that allows the user to directly manipulate four-dimensional (4D) ICE images, thereby eliminating the need for a separate operator to perform the imaging operations according to the instructions received from the cardiac interventionist. In conventional setups, the interventionist must verbally instruct a sonographer to adjust imaging views, whereas this device may allow the cardiac interventionist to perform this independently. Examples of ICE systems and methods described herein avoid the need for verbal instruction between the interventionist and the sonographer by embedding a programmable interface with a plurality of buttons on the ICE catheter handle, allowing the user—in this case, the cardiac interventionist—to select different viewing modes and control directional or imaging context.

The ICE catheter handle can be connected (e.g., wirelessly or with a wired connection) to an imaging or processing system, ensuring seamless integration into existing medical ultrasound devices. In some embodiments, the ICE catheter handle can include electronic circuitry for all, or at least some, of the processing of data received from the catheter. In some embodiments, the ICE catheter handle can include electronic circuitry for all, or at least some, of the control functions of the catheter and the imaging provided by the catheter (e.g., by an array of ultrasound transducers at the distal end of the catheter). Incorporating imaging controls on the ICE catheter handle enables real-time, physician-controlled manipulation of volumetric imaging planes, including plane rotation, forward and backward translation, and depth adjustments. Additionally, configurable movement constraints allow users to tailor workflow preferences, while on-screen indicators provide real-time visual feedback on the current imaging plane position and movement.

By reducing the need for third-party imaging control, the disclosed devices may significantly enhance procedural efficiency, improve physician autonomy, and reduce reliance on external imaging personnel. The ability to manipulate volumetric imaging in real time directly from the ICE catheter handle may facilitate clinical decision-making, reduce operating room clutter, and increase precision in ICE-guided interventions. The disclosed systems, methods, and devices may contribute to a more streamlined, efficient, and physician-driven approach to real-time intracardiac imaging.

In some examples, a catheter handle can include: a housing; a catheter interface coupled to the housing and configured to mechanically and electrically couple to an imaging catheter; and a control interface disposed on the housing, the control interface including: a first actuator configured to move a plane imaged by the imaging catheter in a first direction when the imaging catheter is coupled with the catheter interface; a second actuator configured to move the plane imaged by the imaging catheter in a second direction when the imaging catheter is coupled with the catheter interface; and a third actuator configured to rotate the plane imaged by the imaging catheter when the imaging catheter is coupled with the catheter interface.

In some examples, the control interface further includes a fourth actuator configured to change an imaging mode of the imaging catheter when the imaging catheter is coupled with the catheter interface. In some examples, the catheter interface is configured to couple with an intracardiac echocardiography catheter. In some examples, the control interface is configured to change a position of the plane imaged with real-time volumetric ultrasound imaging in real-time. In some examples, the first actuator and the second actuator include a circular button having four directional inputs corresponding to upward, downward, left, and right movement of the plane. In some examples, at least one of the third actuator or the fourth actuator includes a plurality of auxiliary buttons radially positioned around the first actuator. In some examples, the fourth actuator is configured to switch the imaging mode to at least one of B-Mode, Color Doppler, or Pulse-Wave Doppler. In some examples, the catheter handle can include a fifth actuator configured to freeze or unfreeze an image taken by the imaging catheter when the imaging catheter is coupled with the catheter interface. In some examples, the first actuator and the second actuator are configured to translate a vertical plane within a volumetric space. In some examples, the first actuator includes a plurality of buttons, wherein individual buttons of the plurality of buttons are configured to move different planes within a volumetric space.

In some examples, a system can include: an imaging catheter; and a catheter handle including: a housing; a catheter interface coupled to the housing and configured to mechanically and electrically couple to the imaging catheter; and a control interface disposed on the housing, the control interface including: a first actuator configured to move a plane imaged by the imaging catheter in a first direction when the imaging catheter is coupled with the catheter interface; and a second actuator configured to move the plane imaged by the imaging catheter in a second direction when the imaging catheter is coupled with the catheter interface.

In some examples, the control interface further includes: a third actuator configured to rotate the plane imaged by the imaging catheter when the imaging catheter is coupled with the catheter interface; and a fourth actuator configured to change an imaging mode of the imaging catheter when the imaging catheter is coupled with the catheter interface. In some examples, the imaging catheter includes an intracardiac echocardiography catheter. In some examples, the control interface is configured to change a position of the plane imaged with real-time volumetric ultrasound imaging in real-time. In some examples, the first actuator and the second actuator include a circular button having four directional inputs corresponding to upward, downward, left, and right movement of the plane. In some examples, at least one of the third actuator or the fourth actuator includes a plurality of auxiliary buttons radially positioned around the first actuator. In some examples, the fourth actuator is configured to switch the imaging mode to at least one of B-Mode, Color Doppler, or Pulse-Wave Doppler. In some examples, the system can include a fifth actuator configured to freeze or unfreeze an image taken by the imaging catheter when the imaging catheter is coupled with the catheter interface. In some examples, the first actuator and the second actuator are configured to translate a vertical plane within a volumetric space. In some examples, the first actuator includes a plurality of buttons, wherein individual buttons of the plurality of buttons are configured to move different planes within a volumetric space.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of an example ICE catheter handle with a programmable control interface positioned on a top surface of the handle (e.g., opposite a base section), wherein the programmable control interface includes a plurality of buttons configured to control imaging during a medical procedure, such as a 4D ICE imaging procedure.

FIG. 2 is a schematic of the example of a programmable control interface buttons illustrated in FIG. 1.

FIG. 3 is an illustration showing an example of a vertical shift of a horizontal imaging plane along the z-axis which can be controlled by the programmable control interface.

FIG. 4 is a schematic showing the rotation about the y-axis of the horizontal plane via the buttons embedded on the ICE catheter handle.

FIG. 5 is a schematic showing the shift along the x-axis of the vertical plane via the buttons embedded on the ICE catheter handle.

FIG. 6. Illustrates an instance of the simultaneous scanning of up to three planes within the volumetric space using the data received from the catheter.

FIG. 7 illustrates an example of a user-interface screen displaying the visual results of the ICE scan.

### DETAILED DESCRIPTION

The present disclosure relates to intracardiac echocardiography (ICE) systems and, more specifically, to an ICE catheter handle incorporating integrated controls that enable real-time manipulation of four-dimensional (4D) volumetric ultrasound imaging directly from the handle during a procedure. Disposable catheters have been used in treating cardiovascular, urological, gastrointestinal, neurovascular, and ophthalmic diseases. A catheter modality, as used herein, is a broad term that generally refers to catheters, components, and processing equipment and software associated with a particular procedure that uses a particular catheter. In treating cardiovascular diseases, there are at least four catheter modalities, including intracardiac echocardiography (ICE), intravascular ultrasound (IVUS), radiofrequency (RF) ablation, and fractional flow reserve (FFR). Each modality typically involves catheters and associated components, processing equipment, and software specific to that modality.

Intracardiac echocardiography (ICE) uses an ultrasound array to generate imaging data that provides real-time, direct visualization of anatomical cardiac structures in a heart during a medical procedure. ICE facilitates medical procedures, such as radiofrequency (RF) ablation, by providing real-time visualization of cardiac tissue and structures during a procedure. ICE imaging can be performed in two-dimensional (2D), three-dimensional (3D), or 4D modalities. 2D ultrasound imaging provides real-time, cross-sectional views of the heart and surrounding vasculature. 2D ultrasound imaging relies on a single plane of ultrasound waves to generate grayscale images, offering high-resolution anatomical details but limited spatial context. 3D ultrasound imaging expands upon 2D imaging by acquiring volumetric datasets, enabling enhanced spatial representation of cardiac structures. 3D ICE imaging captures volumetric datasets that reconstruct intracardiac anatomy, improving procedural accuracy by restoring depth perception and facilitating the assessment of complex cardiac geometries. 3D imaging is particularly advantageous in guiding catheter-based interventions, such as ablation procedures and structural heart repairs. 4D ultrasound imaging extends 3D imaging by incorporating real-time temporal resolution, allowing dynamic visualization of cardiac motion. 4D ICE imaging enables clinicians to observe the heart’s structural changes over time, significantly improving procedural guidance and assessment of functional parameters. The integration of 4D imaging in ICE enhances precision in electrophysiological procedures, reducing the reliance on fluoroscopy and reducing radiation exposure. These advancements in ultrasound technology contribute to improved safety, procedural efficiency, and diagnostic accuracy in intracardiac applications, particularly in electrophysiology and interventional cardiology.

In intracardiac echocardiography, three primary imaging modes—B-mode, Color Doppler, and Pulse-Wave Doppler—provide complementary insights into cardiac anatomy and blood flow. B-mode, or brightness mode, is the fundamental grayscale imaging technique that offers high-resolution, real-time visualization of cardiac structures, catheter positioning, and surrounding anatomy, making B-mode imaging useful for guiding interventional procedures. Color Doppler overlays a color map on the B-mode image to depict blood flow direction and velocity, using red and blue hues to indicate flow toward or away from the transducer. Color Doppler imaging is particularly useful for identifying intracardiac shunts, valve regurgitations, and abnormal flow patterns. Pulse-Wave Doppler, on the other hand, enables precise measurement of blood flow velocity at specific locations by placing a Doppler gate at a chosen depth, allowing for detailed assessment of inflow and outflow velocities, diastolic function, and hemodynamic conditions. While B-mode provides essential anatomical details, Color Doppler offers a qualitative assessment of blood flow, and Pulse-Wave Doppler delivers quantitative velocity measurements. Together, B-mode, Color Doppler, and Pulse-Wave Doppler imaging enhance the accuracy of ICE in both diagnostic and procedural applications.

Generally, performing a procedure using a catheter modality comprises two systems. A first system can include a hardware unit having electronics and embedded software configured to send control signals, receive data, process the data, and execute display and operation functions. The hardware unit can include one or more components (e.g., computer(s), display screen(s), etc.). A second system can include a catheter having, for example, a catheter tip with a functional device (e.g., an imaging device, a sensor, an ablation device, etc.), and a catheter handle that is configured to couple to the catheter and operate the catheter. The catheter handle may be in communication, via a wired or wireless connection, with the hardware unit such that the catheter is also in communication with the hardware unit. Normally, the hardware unit is placed on a wheeled platform so that the apparatus can be moved around a room or easily transported to different places (e.g., a procedure room or a storage room). Different catheter modalities have been developed and are used across distinct procedures to treat a range of diseases. Even when using a single catheter modality, execution of the modality can include different apparatuses and systems developed by different suppliers. As a result, each catheter modality typically operates within its own hardware unit (and associated components) and a corresponding catheter system (and associated components), and catheter modalities are generally not configured to share hardware or components.

One of the primary challenges in current 4D ICE imaging methodologies is the limited ability of the cardiac interventionist to directly manipulate and control the imaging system during a procedure. At present, image adjustments are typically performed remotely by a separate sonographer, who operates the imaging console based on verbal instructions from the interventionist. This indirect control mechanism introduces inefficiencies, disrupts procedural workflow, and increases the likelihood of incorrect operation of the imaging system due to miscommunication. The necessity for continuous verbal coordination between the interventionist and the sonographer prolongs procedures and constrains the interventionist’s ability to make immediate, image-informed clinical decisions.

Moreover, the physical environment of an operating room presents additional challenges. Operating rooms are often compact spaces occupied by multiple medical practitioners, technicians, and an array of medical equipment. The presence of numerous cables, devices, and personnel can create spatial constraints and potential safety hazards. The reliance on a remote imaging operator further complicates the workflow, requiring additional coordination that could otherwise be streamlined if the interventionist had direct control over imaging adjustments.

### ICE Catheter Handle With 4D ICE Control

Embodiments disclosed herein describe systems, methods, and processes for manipulating 4D ICE images generated during the procedure directly from the catheter handle by the cardiac interventionist without the need for a third-party operator. The control system embedded on the ICE catheter handle may consist of a programmable interface with multiple control buttons. These buttons may enable the user—typically an interventionist—to manipulate the imaging data by selecting different imaging modes (e.g., B-Mode, color Doppler, and Pulse-Wave), adjust imaging planes by shifting or rotating them, and otherwise control directional or imaging context settings. In contrast to traditional disposable ICE catheter handles that generally lack electronic components, the ICE catheter handle described herein can be a durable and reusable device specifically designed and manufactured to support effective sterilization.

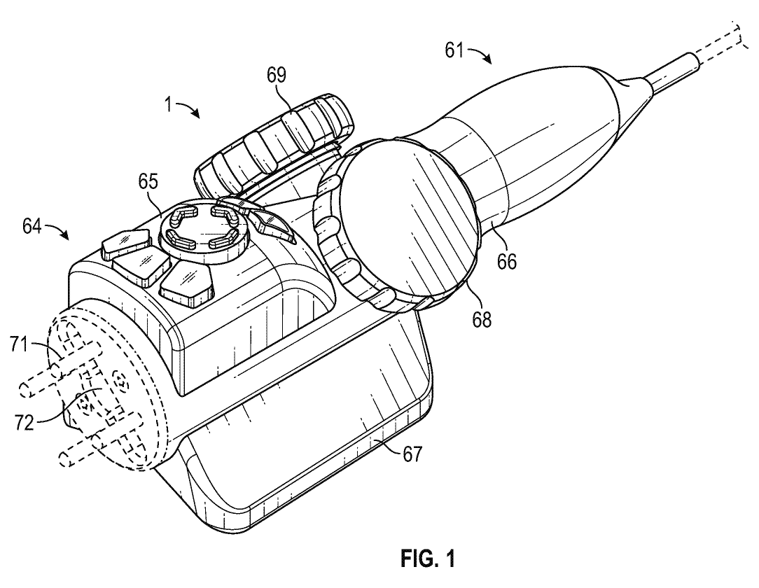

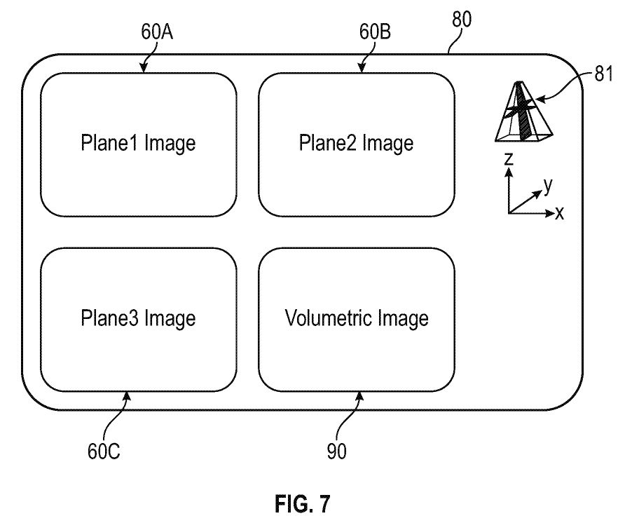

FIG. 1 is a perspective view of an example embodiment of a durable ICE catheter handle 1, which can be used with an ICE catheter. In this example, the ICE catheter handle 1 also includes a programmable control interface 65 that allows the interventionist to perform imaging operations directly from the ICE catheter handle 1. The ICE catheter handle 1 may also have a housing 66 that has a proximal end 61 and a distal end 64. The ICE catheter handle 1 may include, on its distal end 64, a catheter interface including a mechanical interface 71 and/or an electrical interface 72 which can be configured to mechanically and/or electrically couple to a catheter. The electrical interface 72 can transmit control signals to a component of the catheter—such as an ultrasound transducer array—to govern transmit/receive parameters (e.g., beamforming, pulse sequencing, and acquisition settings). The electrical interface 72 can also receive raw or preprocessed imaging data from the ultrasound array component for communication to the processing unit. The mechanical interface may be coupled to the catheter to enable catheter steering. For example, the ICE catheter handle 1 may be coupled to a base 67 that stabilizes the handle and prevents rotation when the base 67 is placed on a surface, such as a portion of a patient. In FIG. 1, the ICE catheter handle 1 also includes a first actuator 68 and a second actuator 69 that may be configured to couple with an attached catheter, and steer the catheter inside the patient’s body. In some embodiments, the programmable control interface 65 may be embedded on the handle 1 and can be connected via wires or wirelessly to a user-interface screen 80 (FIG. 7) where the 4D ICE generated images can be displayed. In some embodiments, the system may be designed to integrate with existing ICE imaging platforms, enhancing compatibility with modern ultrasound processing units. In some embodiments, the control interface 65 may be positioned at alternative locations on the ICE catheter handle 1 to improve ergonomics, accommodate handle geometry, and change the button layout.

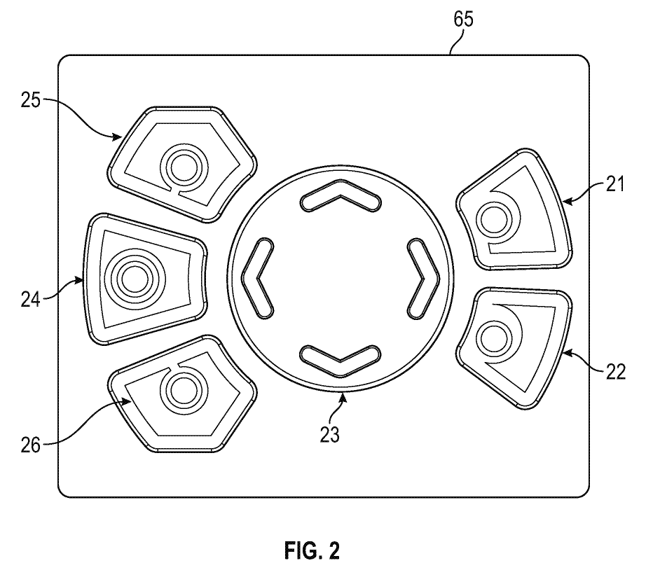

FIG. 2 illustrates an example of a schematic of a programmable control interface 65 that is included on the ICE catheter handle 1 illustrated in FIG. 1, in accordance with one or more embodiments. In this example of a programmable control interface, a multi-directional control 23 is a button that is circular in shape with four directional arrows aligned in a forward, back, left and right direction indicating upward, downward, left, and right directional movements. Additionally, a plurality of auxiliary buttons 21, 22, 24, 25, and 26 are radially positioned around the multi-directional control 23 and can perform various functions. In some embodiments, any one of the auxiliary buttons can be programmed to perform one or more different functions. In some embodiments, one or more of the auxiliary buttons 21, 22, 24, 25, and 26 may also be configured to recognize different input types, such as short, rapid, or prolonged presses, to enable distinct functionalities. In an example: (i) auxiliary button 25 may be programmed to switch between different imaging modes—B-Mode, Color Doppler, and Pulse-Wave Doppler—with a short press and to enter the 4D multi-plane control mode with a long press; (ii) auxiliary button 24 may be programmed to control the imaging depth and the sector angle of the ultrasound field of view; (iii) auxiliary button 26 may be programmed to perform gain control (e.g., increase or decrease overall image brightness and sensitivity to echoes in the selected image mode); (iv) auxiliary button 21 may be programmed to freeze or unfreeze an image; and (v) auxiliary button 22 may be programmed to save a still image if the image is frozen, and to save a video loop if the image is not frozen. In some examples, the multi-directional control 23 is context-sensitive. In B-mode, the up and down arrows of the multi-directional control 23 may adjust imaging depth, and the left and right arrows may adjust the sector angle. In Color Doppler mode, the arrows of the multi-directional control 23 may reposition the Color Doppler box. In Gain mode, the up and down arrows of the multi-directional control 23 may adjust gain for the active imaging mode—B-mode gain when B-mode is active, or Color Doppler gain when Color Doppler is active.

In some embodiments, the auxiliary buttons 21, 22, 24, 25, and 26 may also be configured to recognize different input types, such as short, rapid, or prolonged presses, to enable distinct functionalities. For instance, a quick press may allow incremental plane rotation, while a sustained press may facilitate smooth and continuous rotation. Additionally, the auxiliary buttons 21, 22, 24, 25, and 26 may be programmed to indicate active imaging planes or enable image review within a volumetric space. Furthermore, in some embodiments, the multi-directional control 23 directional buttons may be programmed to provide context-sensitive directional or imaging control, allowing for precise manipulation of imaging parameters.

In some embodiments, the number of auxiliary buttons may be more or less than what is shown in FIG. 2. Furthermore, in some embodiments, the shape and the position of the buttons 21, 22, 23, 24, 25, and 26 may be different than what is shown in FIG. 2.

In some embodiments, the programmable control interface 65 includes LED-backlit buttons that provide status feedback—for example, indicating which imaging plane is currently active or being adjusted. This feature enables the user to immediately identify the imaging plane being adjusted, thereby reducing cognitive load and reducing the risk of unintended modifications. Furthermore, in some embodiments, the system may include on-screen graphical indicators, providing a visual representation of the handle’s controls and their corresponding effects on image planes. This feedback mechanism may enhance user interaction by allowing the interventionist to correlate hand movements with imaging adjustments, eliminating the need to divert attention from the procedure. Additionally, in some embodiments, the physical buttons 21, 22, 23, 24, 25, and 26 of the programmable control interface 65 may be replaced with a digital interface that performs the same functions.

In some embodiments, the ICE catheter handle 1 may be designed with an ergonomic layout of the programmable control interface 65, allowing seamless interaction with 4D ICE imaging while maintaining a natural grip for the interventionist.

In some embodiments, the auxiliary buttons 21, 22, 23, 24, 25, and 26 may be programmed to indicate active imaging planes or enable image review within a volumetric space. Furthermore, in some embodiments, the multi-directional control 23 may be programmed to provide context-sensitive directional or imaging control, allowing for precise manipulation of imaging parameters.

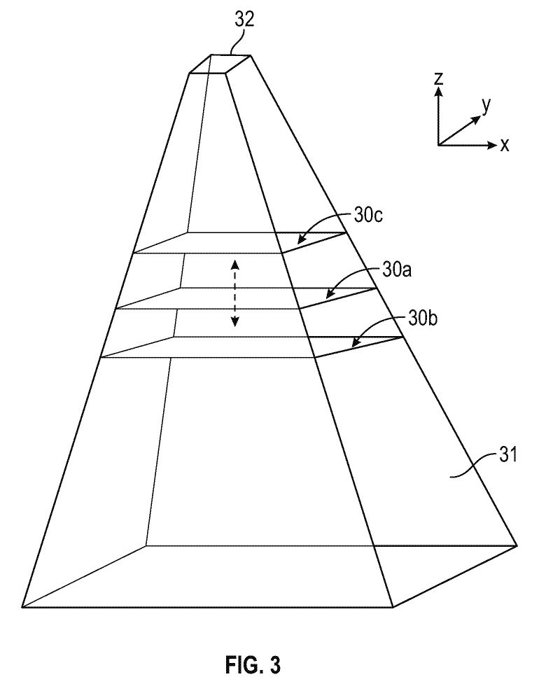

FIG. 3 is a schematic representation illustrating an example of the capability of the ICE catheter handle 1 to adjust the position of the horizontal viewing plane 30a, enabling the scanning of different cross-sections 30b, 30c within a volumetric space 31 generated by an ICE transducer 32. The multi-directional control 23 may be programmed to facilitate the vertical translation of the horizontal viewing plane 30a (the xy-plane) within the volumetric space 31 by, for example, pressing the up and down arrows, which could enable viewing the image of cross-sections 30b, 30c. In some embodiments, a user-interface may be configured to display the movement of the horizontal plane in response to user input from the multi-directional control 23. This visualization may appear within a designated sub-panel 81 (FIG. 7) on the user-interface screen 80 (FIG. 7). Moreover, in some embodiments, some buttons in the programmable control interface 65 may be programmed to provide the user with the option to shift the plane either across the entire volumetric space 31 or within a defined sub-volume, thereby enhancing flexibility in image manipulation. Furthermore, in certain embodiments, the user-interface may enable the configuration of maximum and minimum limits on the translation of the horizontal viewing plane 30a to accommodate the specific requirements of a given procedure.

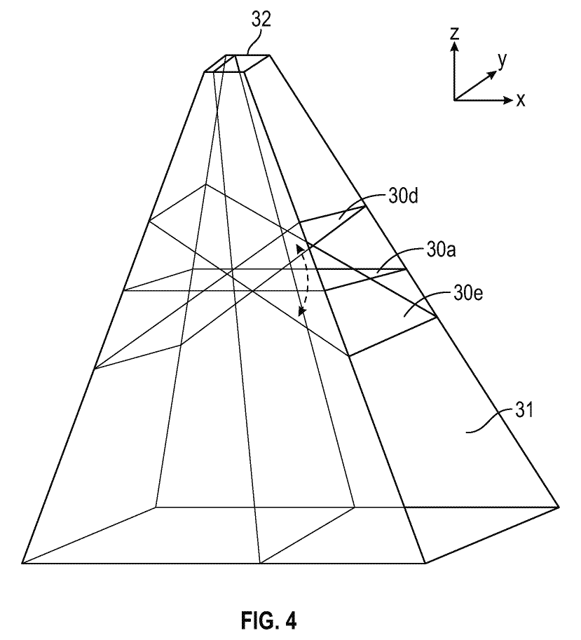

FIG. 4 is a schematic representation illustrating an example of the capability of the ICE catheter handle 1 to rotate the position of the horizontal viewing plane 30a around the y-axis, enabling the scanning of different cross-sections 30d, 30e within a volumetric space 31 generated by an ICE transducer 32. The multi-directional control 23 may be programmed to facilitate the rotation of the horizontal viewing plane 30a (xy-plane) around the y-axis within the volumetric space 31 by, for example, pressing the right and left arrow buttons which could enable viewing the image of cross-sections 30d, 30e of the volumetric space 31. In some embodiments, the user-interface may be configured to display the movement of the horizontal viewing plane 30a in response to user input from the multi-directional control 23 within a designated sub-panel 81 on the user-interface screen 80. Furthermore, in certain embodiments, the user-interface may enable the configuration of maximum and minimum limits on the degree of rotation to accommodate the specific requirements of a given procedure. For example, in some embodiments the system may impose a maximum plane rotation limit of 89 degrees to prevent unintended plane transitions, thereby enhancing procedural precision and stability. This constraint may be implemented because rotating a plane by 90 degrees about an axis changes its identity—for example, rotating the xy-plane by 90 degrees about the y-axis changes it into a representation of the yz-plane. By limiting rotation to a maximum of 89 degrees, the system ensures that planes remain within their intended orientations, preventing unintended transformations that could affect procedural accuracy.

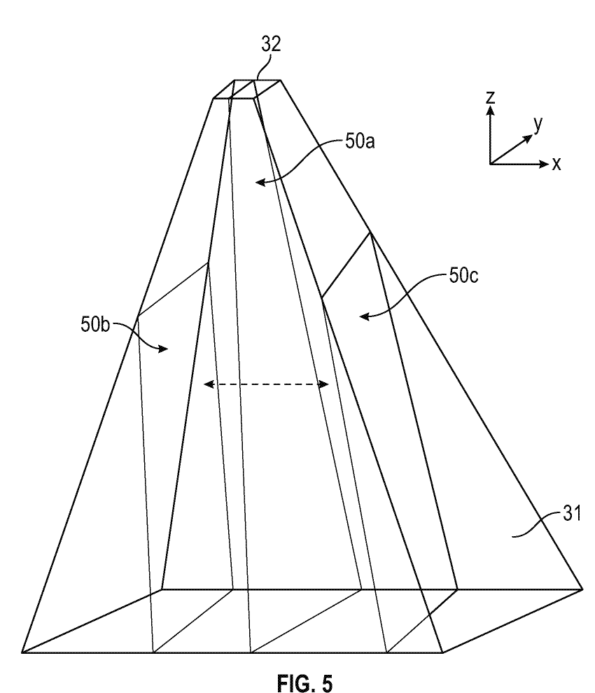

FIG. 5 is a schematic representation illustrating an example of a capability of the ICE catheter handle 1 to adjust the position of the vertical viewing plane 50a, enabling the scanning of different cross-sections 50b, 50c within a volumetric space 31 generated by an ICE transducer 32. The multi-directional control 23 may be programmed to facilitate the horizontal translation of the vertical viewing plane 50a (the yz-plane) within the volumetric space 31 by, for example, pressing the up and down arrows. In some embodiments, the user-interface may be configured to display the movement of the vertical viewing plane 50a in response to user input from the multi-directional control 23 within a designated sub-panel 81 on the screen 80. Moreover, in some embodiments, some buttons in the control system 65 may be programmed to provide the user with the option to shift the plane either across the entire volumetric dataset or within a defined sub-volume, thereby enhancing flexibility in image manipulation. Furthermore, in certain embodiments, the user-interface may enable the configuration of maximum and minimum limits on the translation of the planes to accommodate the specific requirements of a given procedure.

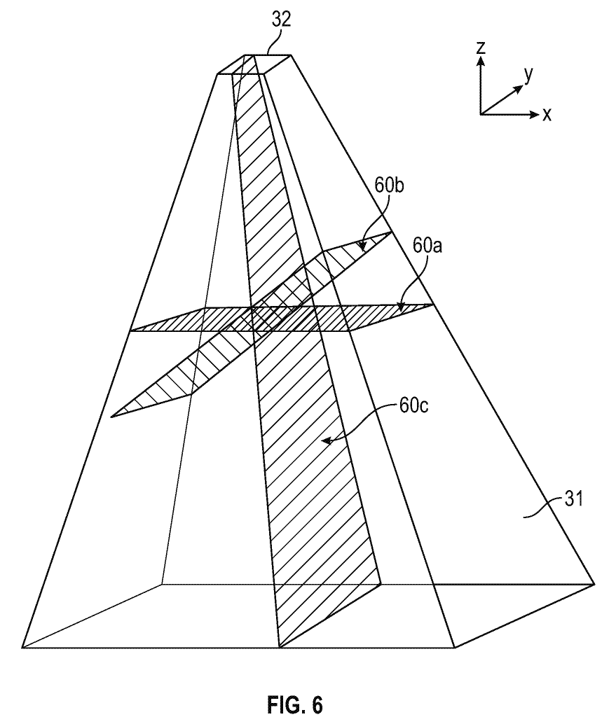

FIG. 6 illustrates the simultaneous scanning of up to three planes within the volumetric space. In this example, the multi-directional control 23 of the ICE catheter handle 1 can enable the concurrent scanning of three distinct planes, shown as example planes 60a, 60b, and 60c, within the volumetric space 31 using the ICE transducer 32. The planes 60a, 60b, and 60c can be aligned at various angles other than what is illustrated in FIG. 6. The resulting imaging data can be transmitted to a user-interface screen 80 where the cross-sectional images corresponding to planes 60a, 60b, and 60c can be displayed to an operator, and the volumetric image 90 can also be displayed. Additionally, the operator can precisely identify the locations of the scanned planes within the volumetric space 31 through a dedicated sub-panel 81 on the user-interface screen 80. The operator may initially commence scanning with the three planes positioned at their default locations, coinciding with the xy, xz, and yz planes. Using the programmable control interface 65 of the ICE catheter handle 1, an operator can independently rotate or translate each plane without affecting the others and observe the result of the manipulation on the user-interface screen 80. As a result of this independent manipulation, in certain embodiments, the scanned cross-sections may deviate from orthogonal orientations and no longer correspond to conventional geometric planes, thereby enabling more flexible and customized imaging. Additionally, in some embodiments, the planes are color-coded for convenience of the operator to precisely distinguish the planes and the resulting images.

FIG. 7 illustrates an example of a user-interface screen displaying the visual results of the ICE scan. In this example, the user-interface screen 80 comprises four distinct panels (or windows) each dedicated to visualizing the three separate scanning planes 60a, 60b, and 60c along with the corresponding volumetric image 90. Additionally, in this example the user-interface screen 80 includes a sub-panel 81 that dynamically visualizes the planes, reflecting their rotation and translation in response to the operator’s input via the programmable control interface 65.

In some embodiments, the user-interface screen 80 may be programmed to display additional imaging results in accordance with various imaging modes, including B-Mode, Color Doppler, and Pulse-Wave Doppler. Furthermore, the sub-panel 81 may also be configured to provide additional visualizations related to the scanned planes, adapting to different imaging modes as required.

Moreover, in certain embodiments, the user-interface screen 80 may be divided into a customizable number of sub-panels, either for user convenience or at the operator’s discretion. Additionally, in some embodiments, the user-interface screen 80 may incorporate a touchscreen, enabling interactive manipulation of the image results, such as decreasing or increasing image size, as well as zooming in or out to enhance visualization and analysis.

### Example Implementations

Examples of the implementations of the present disclosure are described in view of the following example clauses. The features recited in the below example implementations are combinable with additional features disclosed herein. Furthermore, additional inventive combinations of features are disclosed herein, which are not specifically recited in the below example implementations, and which do not include the same features as the specific implementations below. For sake of brevity, the below example implementations do not identify every inventive aspect of this disclosure. The below example implementations are not intended to identify key features or essential features of any subject matter described herein. Any of the example clauses below, or any features of the example clauses, are combinable with any one or more other example clauses, or features of the example clauses or other features of the present disclosure.

Example 1. A catheter handle comprising: a housing having a longitudinal axis, the housing having: a distal end and a proximal end; an interface on the distal end for mechanically and electrically coupling to an intracardiac echocardiography (ICE) catheter; and a control interface comprising a plurality of controls, the control interface disposed on the housing and configured to provide control of imaging functionality of an ICE catheter coupled to the ICE catheter handle.

Example 2. The ICE catheter handle of example 1, wherein the control interface includes a multi-directional control button and a plurality of auxiliary buttons disposed around the multi-directional control button.

Example 3. The ICE catheter handle of example 1, further comprising a base coupled to a bottom portion of the housing, wherein the control interface is positioned on a top portion of the housing.

Example 4. The ICE catheter handle of example 1, further comprising a first and second actuator configured to mechanically couple to a catheter and control movement of the catheter in two orthogonally planes, wherein the control interface is positioned between the first and second actuators and the distal end of the housing such that it can be easily accessed by a user controlling the first and second actuators.

Example 5. The ICE catheter handle of example 1, wherein the control interface is configured to rotate an image plane around a y-axis in a volume generated by an ICE catheter coupled to the handle.

Cluse 6. The ICE catheter handle of example 5, wherein the control interface is configured to have a minimum and/or maximum rotation angle of the image plane.

Example 7. The ICE catheter handle of example 6, where the minimum and maximum rotation angle in between about 1° and about 89°.

Example 8. The ICE catheter handle of example 1, where the control interface is configured to control shifting a plane imaged by an ultrasound element of the catheter in a volumetric space.

Example 9. The ICE catheter handle of example 5, where the minimum and maximum translation limit is set by the operator.

Example 10. The catheter of example 5, where the control buttons may be programmed to provide the user with the option to shift the plane either across the entire volumetric dataset or within a defined sub-volume

Example 11. The ICE catheter handle of example 1, where one or more buttons disposed on the housing are configured to cause the user-interface screen to toggle between different ICE imaging modes, including B-Mode, color Doppler, and Pulse-Wave Doppler.

Example 12. The ICE catheter handle of example 1, where one or more buttons disposed on the housing are configured to cause the user-interface screen to switch between 2D and 3D imaging modes.

Example 13. The ICE catheter handle of example 1, where one or more buttons disposed on the housing are configured to cause the user-interface screen to freeze or unfreeze the ultrasound image.

Example 14. The ICE catheter handle of example 1, where one or more buttons disposed on the housing are configured to save an image captured by the ultrasound unit in a data processing unit.

Example 15. The ICE catheter handle of example 1, where one or more buttons disposed on the housing are configured to recognize different types of haptic input, including rapid press and prolonged press, in order to perform various functions based on the haptic input.

Example 16. The ICE catheter handle of example 1, wherein a control button within the programmable control button interface is configured to initiate image manipulation, wherein the manipulated image is stored as data and transmitted to a user-interface screen for displaying purposes.

Example 17. The ICE catheter handle of example 1, wherein a control button within the programmable control button interface is configured to initiate image manipulation, wherein the manipulated image is stored as data and wirelessly transmitted to a user-interface screen for display.

Example 18. The ICE catheter handle of example 1, wherein a control button within the programmable control button interface is configured to initiate image manipulation, wherein the manipulated image is stored as data and transmitted to a user-interface screen for displaying purposes using wires connecting the data processing unit with the user-interface screen.

Example 19. A catheter handle comprising: a housing; an interface on the housing configured to mechanically and electrically couple to a catheter; and a control interface disposed on the housing and including: a multi-directional control; and at least one auxiliary button, wherein the control interface is configured to control imaging functionality of the coupled catheter including at least one of manipulating an imaging plane by rotation or translation and switching between imaging modes.

Example 20. The catheter handle of example 19, wherein the catheter comprises an intracardiac echocardiography catheter.

Example 21. The catheter handle of example 19, wherein the imaging functionality comprises real-time volumetric ultrasound imaging.

Example 22. The catheter handle of example 19, wherein the multi-directional control comprises a circular button having four directional inputs corresponding to upward, downward, left, and right movement of an imaging parameter.

Example 23. The catheter handle of example 19, wherein the at least one auxiliary button comprises a plurality of auxiliary buttons radially positioned around the multi-directional control.

Example 24. The catheter handle of example 23, wherein at least one auxiliary button is programmed to switch between imaging modes including B-Mode, Color Doppler, and Pulse-Wave Doppler.

Example 25. The catheter handle of example 24, wherein a short press of the at least one auxiliary button switches between imaging modes and a long press enters a 4D multi-plane control mode.

Example 26. The catheter handle of example 22, wherein the multi-directional control is context-sensitive to provide directional or imaging control based on an active imaging mode.

Example 27. The catheter handle of claim 26, wherein in B-mode the up and down arrows adjust imaging depth and the left and right arrows adjust sector angle.

Example 28. The catheter handle of example 26, wherein in Color Doppler mode the arrows reposition a Color Doppler box.

Example 29. The catheter handle of example 19, wherein at least one auxiliary button is programmed to control imaging depth and sector angle of an ultrasound field of view.

Example 30. The catheter handle of example 19, wherein at least one auxiliary button is programmed to perform gain control.

Example 31. The catheter handle of example 19, wherein at least one auxiliary button is programmed to freeze or unfreeze an image.

Example 32. The catheter handle of example 31, wherein at least one auxiliary button is programmed to save a still image if the image is frozen and to save a video loop if the image is not frozen.

Example 33. The catheter handle of example 19, wherein at least one of the buttons recognizes different input types including short, rapid, or prolonged presses to enable distinct functionalities.

Example 34. The catheter handle of example 19, wherein the control interface is configured to vertically translate a horizontal plane within a volumetric space to view different cross-sections.

Example 35. The catheter handle of example 34, wherein the multi-directional control inputs corresponding to up and down facilitate the vertical translation of the horizontal plane.

Example 36. The catheter handle of example 19, wherein the control interface is configured to rotate a horizontal plane around a y-axis within a volumetric space.

Example 37. The catheter handle of example 36, wherein directional inputs corresponding to left and right facilitate rotation of the horizontal plane around the y-axis.

Example 38. The catheter handle of example 36, wherein the catheter handle is configured to impose a maximum plane rotation limit of 89 degrees to prevent unintended plane transitions.

Example 39. The catheter handle of example 19, wherein the control interface is configured to translate a vertical plane within a volumetric space.

Example 40. The catheter handle of example 39, wherein directional inputs corresponding to up and down facilitate horizontal translation of the vertical plane.

Example 41. The catheter handle of example 19, wherein translation of a plane is limited by operator-selectable minimum and maximum limits.

Example 42. The catheter handle of example 19, wherein shifting of a plane is selectable across an entire volumetric dataset or within a defined sub-volume.

Example 43. The catheter handle of example 19, wherein the control interface enables simultaneous scanning of up to three planes within a volumetric space.

Example 44. The catheter handle of example 43, wherein each of the three planes is independently rotated or translated without affecting the others.

Example 45. The catheter handle of example 43, wherein the planes are color-coded to distinguish the planes and resulting images.

Example 46. The catheter handle of example 19, further comprising a user-interface screen configured to display cross-sectional images and a volumetric image and a sub-panel that visualizes plane positions and movement in response to input from the control interface.

Example 47. The catheter handle of example 46, wherein the user-interface screen comprises four distinct panels each dedicated to visualizing three separate scanning planes and a volumetric image, and a sub-panel configured to reflect rotation and translation of the planes.

Example 48. The catheter handle of example 46, wherein communication between the catheter handle and the user-interface screen is via a wired or a wireless connection.

Example 49. The catheter handle of example 19, wherein the programmable control interface includes LED-backlit buttons configured to provide status feedback indicating which imaging plane is currently active or being adjusted.

Example 50. A method of controlling imaging functionality of a catheter, comprising: providing a catheter handle having a housing; an interface on the housing configured to mechanically and electrically couple to a catheter; and a control interface disposed on the housing including: a multi-directional control; and at least one auxiliary button; coupling the catheter to the interface; and operating the control interface to manipulate an imaging plane by rotation or translation and to switch between imaging modes of the coupled catheter.

Example 51. A system comprising: a catheter; and a catheter handle, the catheter handle including: a housing; an interface on the housing configured to mechanically and electrically couple to the catheter; and a control interface disposed on the housing and including: a multi-directional control; and at least one auxiliary button, wherein the control interface is configured to control imaging functionality of the coupled catheter including manipulating an imaging plane by rotation or translation and switching between imaging modes.

### Terminology

While certain embodiments of the disclosure have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present disclosure is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the systems and methods of the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z. Additionally, the phrase “based on” does not mean “based only on,” unless expressly specified otherwise. In other words, the phrase “based on” describes both “based only on” and “based at least on.” Unless otherwise explicitly stated, articles such as “a” or “an” should generally be interpreted to include one or more described items. Accordingly, phrases such as “a device configured to” are intended to include one or more recited devices.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that throughout the application, data is provided in a number of different formats, and that this data, may represent endpoints or starting points, and ranges for any combination of the data points. For example, if a particular data point “10” and a particular data point “15” may be disclosed, it is understood that greater than, greater than or equal to, less than, less than or equal to, and equal to 10 and 15 may be considered disclosed as well as between 10 and 15. It is also understood that each unit between two particular units may be also disclosed. For example, if 10 and 15 may be disclosed, then 11, 12, 13, and 14 may be also disclosed.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

The foregoing description is that of certain features, aspects, and advantages of the devices, systems, and methods, to which various changes and modifications can be made without departing from the spirit and scope of the devices, systems, and methods. Moreover, the devices described herein need not feature all of the objects, advantages, features, and aspects discussed above. Thus, for example, those of skill in the art will recognize that the devices, systems, and methods can be embodied or carried out in a manner that achieves or optimizes one advantage or a group of advantages as taught herein without necessarily achieving other objects or advantages as may be taught or suggested herein. In addition, while a number of variations of the devices, systems, and methods have been shown and described in detail, other modifications, and methods of use, which are within the scope of the devices, systems, and methods, will be readily apparent to those of skill in the art based upon this disclosure. It is contemplated that various combinations or subcombinations of these specific features and aspects of embodiments may be made and still fall within the scope of the devices, systems, and methods disclosed herein. Accordingly, it should be understood that various features and aspects of the disclosed embodiments can be combined with or substituted for one another in order to form varying modes of the discussed devices.

What is claimed is:

1. A catheter handle comprising:
a housing;
a catheter interface coupled to the housing and configured to mechanically and electrically couple to an imaging catheter; and
a control interface disposed on the housing, the control interface comprising:
a first actuator configured to move a plane imaged by the imaging catheter in a first direction when the imaging catheter is coupled with the catheter interface;
a second actuator configured to move the plane imaged by the imaging catheter in a second direction when the imaging catheter is coupled with the catheter interface; and
a third actuator configured to rotate the plane imaged by the imaging catheter when the imaging catheter is coupled with the catheter interface.

2. The catheter handle of claim 1, wherein the control interface further comprises a fourth actuator configured to change an imaging mode of the imaging catheter when the imaging catheter is coupled with the catheter interface.

3. The catheter handle of claim 1, wherein the catheter interface is configured to couple with an intracardiac echocardiography catheter.

4. The catheter handle of claim 1, wherein the control interface is configured to change a position of the plane imaged with real-time volumetric ultrasound imaging in real-time.

5. The catheter handle of claim 1, wherein the first actuator and the second actuator comprise a circular button having four directional inputs corresponding to upward, downward, left, and right movement of the plane.

6. The catheter handle of claim 2, wherein at least one of the third actuator or the fourth actuator comprises a plurality of auxiliary buttons radially positioned around the first actuator.

7. The catheter handle of claim 2, wherein the fourth actuator is configured to switch the imaging mode to at least one of B-Mode, Color Doppler, or Pulse-Wave Doppler.

8. The catheter handle of claim 1, further comprising a fifth actuator configured to freeze or unfreeze an image taken by the imaging catheter when the imaging catheter is coupled with the catheter interface.

9. The catheter handle of claim 1, wherein the first actuator and the second actuator are configured to translate a vertical plane within a volumetric space.

10. The catheter handle of claim 1, wherein the first actuator comprises a plurality of buttons, wherein individual buttons of the plurality of buttons are configured to move different planes within a volumetric space.

11. A system for imaging comprising:
an imaging catheter; and
a catheter handle comprising:
a housing;
a catheter interface coupled to the housing and configured to mechanically and electrically couple to the imaging catheter; and
a control interface disposed on the housing, the control interface comprising:
a first actuator configured to move a plane imaged by the imaging catheter in a first direction when the imaging catheter is coupled with the catheter interface; and
a second actuator configured to move the plane imaged by the imaging catheter in a second direction when the imaging catheter is coupled with the catheter interface.

12. The system of claim 11, wherein the control interface further comprises:
a third actuator configured to rotate the plane imaged by the imaging catheter when the imaging catheter is coupled with the catheter interface; and
a fourth actuator configured to change an imaging mode of the imaging catheter when the imaging catheter is coupled with the catheter interface.

13. The system of claim 11, wherein the imaging catheter comprises an intracardiac echocardiography catheter.

14. The system of claim 11, wherein the control interface is configured to change a position of the plane imaged with real-time volumetric ultrasound imaging in real-time.

15. The system of claim 11, wherein the first actuator and the second actuator comprise a circular button having four directional inputs corresponding to upward, downward, left, and right movement of the plane.

16. The system of claim 12, wherein at least one of the third actuator or the fourth actuator comprises a plurality of auxiliary buttons radially positioned around the first actuator.

17. The system of claim 12, wherein the fourth actuator is configured to switch the imaging mode to at least one of B-Mode, Color Doppler, or Pulse-Wave Doppler.

18. The system of claim 11, further comprising a fifth actuator configured to freeze or unfreeze an image taken by the imaging catheter when the imaging catheter is coupled with the catheter interface.

19. The system of claim 11, wherein the first actuator and the second actuator are configured to translate a vertical plane within a volumetric space.

20. The system of claim 11, wherein the first actuator comprises a plurality of buttons, wherein individual buttons of the plurality of buttons are configured to move different planes within a volumetric space.
